(19) United States
(12) Patent Application Publication
(10) Pub. No.: US 2013/0079800 A1
(43) Pub. Date: Mar. 28, 2013

(54) METHOD FOR DEBRIDING HUMAN TISSUE

(71) Applicant: C. Edward Eckert, New Kensington, PA (US)

(72) Inventor: C. Edward Eckert, New Kensington, PA (US)

(21) Appl. No.: 13/684,504

(22) Filed: Nov. 24, 2012

Related U.S. Application Data
(63) Continuation-in-part of application No. 13/369,385, filed on Feb. 9, 2012.
(60) Provisional application No. 61/574,526, filed on Aug. 4, 2011.

(51) Int. Cl. A61B 17/3203

(52) U.S. Cl. CPC A61B 17/3203
USPC 606/131; 606/190

(57) ABSTRACT

There is provided a method for debriding human tissue by dissolving a gas in a liquid to form a solution under elevated pressure to saturate that solution with the dissolved gas. The solution is depressurized at or towards ambient pressure for supersaturating the gas in solution. The method provides means for allowing supersaturated solution to infiltrate the human tissue needing to be debrided. The supersaturated dissolved gas solution is activated and the activated gas allowed to expand for releasing energy into the solution at or near the tissue needing to be debrided. That human tissue is exfoliated with released energy from the expanding activated gas.

### CROSS-REFERENCE TO RELATED APPLICATION(S)

This application is a Continuation-In-Part of pending U.S. application Ser. No. 13/369,385, filed on Feb. 9, 2012, itself a perfection of U.S. Provisional Patent Application Ser. No. 61/574,526, filed on Aug. 4, 2011, both disclosures of which are incorporated by reference herein.

### FIELD OF THE INVENTION

The present invention relates to solutions of dissolved gas in liquids, and also to mixtures consisting of a solution of gas, e.g., CO₂, and a dispersion of the gas in a liquid, e.g., water. More specifically, this invention relates to a method that uses a dioxide-rich environment to treat human tissue by the debridement of same. It is appropriate for treating tissue with burns and/or lacerations.

### BACKGROUND OF THE INVENTION

In the medical community, it is generally known that the effect of oxygen on living tissue can be characterized by three regimes: metabolic enhancement (growth accelerator), metabolic inhibition (growth arrest), and toxicity. In the former regime, oxygenated solutions and microbubble suspensions can be used to accelerate the healing and regeneration rate of damaged tissue. Such wounds include cuts, lacerations, sores and burns on the face, arms, legs, torso and roof of the mouth. When wounds begin to heal, fibroblastic cells divide and spread throughout the wound area. The fibroblastic cells produce collagen, an important protein that facilitates healing. Supplying sufficient quantities of oxygen to the wound area significantly enhances fibroblast proliferation. In particular, the fibroblastic cells use amino acids hydroxylated with oxygen to synthesize collagen chains.

In addition to treating wounds, oxygen is frequently used in topical applications for cleaning and revitalizing skin. In facial cleansing, dissolved oxygen and microbubble suspensions can assist in exfoliating dead skin particles from the skin surface. It may also be possible for dissolved oxygen and microbubble suspensions to lighten skin that has been affected by hyperpigmentation. Oxygen can be used to remove toxins, particulates and other occlusions in skin pores. It is possible that oxygen can oxidize oils in the skin pores, thus allowing the pores to become backfilled with water. Once the skin is removed and dried, the pores would be essentially vacant and receptive to infiltration by beneficial lotions and other skin care products. Without the oxidative effects, pores in the skin would remain filled with oil that would require displacement from the pore before lotions could occupy pore volume.

An improvement is skin topography (roughness) has been observed following exposure of the skin to oxygen dissolved in water. Stereoscopic examination of the skin indicates that the peaks that exist in the epidermal layer of the skin have been become smooth; presumably as a result of selectively higher oxidation rates associated with the higher surface area ridges of the skin.

In addition, oxygen can revitalize skin cells by joining with protein molecules to nourish the cells and produce collagen. It is even possible that dissolved oxygen and microbubble suspensions can stimulate hair follicles and consequentially hair growth.

It is an object of the present invention to provide a novel system for dissolving a gas other than oxygen in a liquid. It is another object of this invention to provide a novel system for incorporating large quantities of gas in liquids. It is yet another object of this invention to dissolve large quantities of carbon dioxide in water. It is still another object of this invention to provide large quantities of CO₂ in water to provide a saturated solution of metastable dioxide gas in water solution and create a holdup of microbubbles in suspension within the solution. And still it is yet another object of this invention to use said carbon dioxide/water suspension-solution for health applications relating to human tissue treatments. Finally, it is another object of this invention to produce saturated and hypersaturated gas-liquid solutions that can be stimulated to nucleate gas bubbles and resulting dispersions for purposes of providing benefits for burns and lacerations to human tissue.

### SUMMARY OF THE INVENTION

In accordance with the foregoing objects and advantages, there is provided a method for debriding human tissue by dissolving a gas in a liquid to form a solution under elevated pressure to saturate that solution with the dissolved gas. Then, the solution is depressurized at or towards ambient pressure for supersaturating the gas in solution. The method provides means for allowing supersaturated solution to infiltrate the human tissue needing to be debrided. The supersaturated dissolved gas solution is activated and the activated gas allowed to expand for releasing energy into the solution at or near the tissue needing to be debrided. Finally, that human tissue is exfoliated with released energy from the expanding activated gas.

The gas of choice for such debridement activities is carbon dioxide though it may be accompanied by exposing the human tissue to a solution containing at least one other dissolved gas selected from: oxygen, nitrous oxide, nitric oxide, carbon monoxide and hydrogen sulfide.

### BRIEF DESCRIPTION OF DRAWINGS

Further features, objectives and advantages of the present invention will be made clearer in the following Detailed Description made with reference to the drawings in which:

FIG. 1 is a graph comparing pH versus the number of volumes capable of being dissolved at various pressures of carbon dioxide gas (P_{CO2});

FIG. 2 is a graph plotting free energy (G) versus the radii of bubbles to be nucleated for showing a metastable state; and

FIG. 3 is a flowchart depicted one preferred embodiment for the method steps of this invention.

### DETAILED DESCRIPTION OF PREFERRED EMBODIMENTS

The various means of exposing living tissue to oxygen that have been developed are based on gas envelopment. The earliest approach is to fill a flexible wall enclosure with essentially pure oxygen at atmospheric pressure (“oxygen tent”) to expose tissue to an oxygen concentration that exceeds ambient conditions by a factor of approximately 5. A patient exposed to such levels of oxygen presumably benefit by enhanced oxygen exposure through both inhalation and cutaneous uptake. The resulting dissolved oxygen concentration in the water component of tissue thus exposed at equilibrium will also increase by a multiple of 5 to a concentration of 40 milligrams O₂ per liter of water or corresponding dissolved oxygen (DO) value of 40 mg/l. Such oxygen is available to participate in various bio-chemical processes, including systemic oxygen uptake in the circulatory system. Oxygen permeability of ocular tissue, for example, has been measured by Weissman et al, and appears below for various ocular tissue types:

| | | | | Contact |
|---|---|---|---|---|
| | | | | Lens |
| O₂ Permeability | Endothelium | Epithelium | Stroma | Tears |
| $\frac{{ml}_{O 2} - {cm}^{2}}{{ml}_{tissue} - sec \text{-} mm Hg}$ | 0.53 × 10⁻¹⁰ | 1.88 × 10⁻¹⁰ | 3 × 10⁻¹⁰ | 0.1 × 10⁻¹⁰ |

It can clearly be seen that ocular tissue exposed to elevated oxygen tension (mm Hg) will result in transcutaneous oxygen transport at these reported permeability values.

An improvement to the oxygen tent is to create a chamber capable of being pressurized with pure oxygen to a value greater than atmospheric pressure. These chambers are known as hyperbaric chambers and therapeutically constitute the broad category of Hyperbaric Oxygen Therapy or “HBOT” devices. In such devices, pure oxygen can be pressurized to a practical limit of approximately 3 atmospheres (2,280 mm Hg or 44 psia—absolute) resulting in an equilibrated DO value of 120 mg/l. Obviously equipment expense, safety, lack of portability, and patient tolerance are inherent limitations to HBOT.

Other means to expose tissue to elevated oxygen concentrations have been developed that are all based on envelopment devices that surround the tissue site and provide a gas phase pure oxygen environment. These devices include flexible wall (‘bag”) enclosures operating at near atmospheric pressure and rigid enclosures with tourniquet type and other sealing arrangements capable of operating somewhat above atmospheric pressure. None of the methods and devices intended for local oxygen application provide respiration uptake, although supplemental oxygen could be delivered through a nasal canella or mask. The intent is to expose local tissue to concentrations of gas phase oxygen greater than ambient values, albeit limited by the pressure tolerance capabilities of the enclosure.

The free radical form of oxygen is generally highly reactive and is biologically considered a reactive oxygen species or “ROS”. ROS are formed as a result of normal biological metabolism and can have a detrimental effect on biological organisms, including acting as carcinogens. Cells contain an enzyme known as super oxide dismutase (SOD) that helps provide cellular level protection from ROS. The superoxide ion, O₂₋ is an example of a biological ROS. The presence of free radical ROS depletes cellular SOD and results in vulnerability for cellular damage.

Hydrogen peroxide is another example of an ROS. Hydrogen peroxide is popular source of oxygen used in topical applications and baths. Atomic oxygen with an unpaired electron is easily derived from hydrogen peroxide, or H₂O₂, because an H₂O₂ molecule readily dissociates into water (H₂O) and O⁻, the latter being an ROS. The decomposition of H₂O₂ into water and oxygen free-radicals creates an enriched solution that facilitates dermal contact with oxygen. Hydrogen peroxide is distributed in various grades and concentrations that are specific to certain applications. Solutions of 3% and 6% hydrogen peroxide are commonly sold to consumers who use the solutions to disinfect cuts and clean skin areas. Solutions of 35% hydrogen peroxide are frequently added to spas and hot tubs to disinfect the water. Skin therapists use solutions of 35% hydrogen peroxide in oxygen baths to improve tissue regeneration and remove toxins from the dermis. Some topical creams contain stabilized forms of hydrogen peroxide intended to prevent infections in skin and result in bleaching of the skin.

Despite being a significant source of oxygen, hydrogen peroxide has been the subject of significant controversy when used in skin treatment applications. Some authorities claim that hydrogen peroxide is cytotoxic to human fibroblasts, due to the presence of free-radical oxygen. As a result, some medical professionals recommend additional dilution of hydrogen peroxide solutions to avoid their toxic effects on skin. Authorities also state that hydrogen peroxide reduces white blood cell activity. Still others have found that hydrogen peroxide slows wound healing by drying the wound, which destroys the exudate and leads to necrosis of skin tissue. Dry tissue also makes the wound area prone to bacterial growth and infection. As a result, hydrogen peroxide has drawn some questions as to its suitability for treating skin wounds and burns.

An alternative approach to all gas phase tissue oxygenation methods and object of the present invention is to use a liquid phase solvent capable of dissolving a molecular gas other than oxygen to exceptionally high DO values while in a stable solution and subsequently exposing tissue to this solution for gas uptake. This solvent, in essence becomes a carrier or delivery means for the gas as a dissolved species, at high chemical potential. In HBOT, oxygen dissolution in cutaneous water occurs within the chamber and is limited by the equilibrium value of the oxygen pressure used. Oxygen dissolution and exposure occur simultaneously and in the same environment. Conversely, a very high DO solution is created by this invention that is separate from tissue exposure and operates under conditions most kinetically and energetically favorable for dissolution, without the limitations of HBOT.

This invention relates to solutions having quantities of dissolved gas, and may also involve mixtures containing such a solution having dissolved gas and a dispersion of ultra-small microbubbles of the gas in suspension within the solution; all intended for medical applications. The solvent component of the solution is typically water and the gas solute is typically carbon dioxide, although other gases such as oxygen alone, nitrous oxide, nitric oxide, hydrogen sulfate and combinations thereof, can add medicinal benefits.

When present, microbubbles generally have a diameter of less than about 125 microns, and preferably less than 50 microns, and are defined as such. It is desirable in the context of this invention to minimize the terminal velocity of the microbubbles within the suspending liquid. Since the residence time of a bubble in a liquid is inversely proportional to the square of the bubble diameter, such microbubbles will have an extended contact time with a fluid as compared to a system consisting of larger diameter bubbles. This quality is known as gas hold-up. Additionally, the microbubble/liquid interfacial area is inversely proportional to the square of the bubble diameter. Therefore, it will be shown that a solution of a solute gas such as oxygen in a solvent liquid such as water can be manipulated to produce a dispersion of microbubbles consisting of solute gas nucleated and precipitated from the liquid. This results in an extended residence time and high interfacial area that maximizes the contact between the gas (as microbubbles) and a surface placed within the liquid, such as living tissue.

### Background HSAS Production

The amount of oxygen initially dissolved into solution is largely dependent on the method used to dissolve the oxygen gas. These methods generally consist of two steps: creating a solute gas/solvent liquid interfacial area, and, exposing the gas/liquid mixture to elevated pressure. The former step affects the kinetics or rate at which the solution process occurs while the latter determines the maximum theoretical dissolved oxygen concentration.

In general terms, a solute species to be dissolved into a solvent requires an interface Oxygen as a candidate solute species requires a gas/liquid or, specifically, an oxygen/water interface in order to be transported into the solvent.

Small bubbles create interfacial area and promote more favorable kinetics. The second step is a pressure-concentration relationship, such as Henry's Law for dilute solutions and Sievert's Law for diatomic gases at higher concentrations. These steps can be combined, although the source of oxygen must operate at the higher final pressure rather than allowing a pump, for example, to pressurize both the liquid and gas components after the gas has been introduced.

### Pneumatic Debridement

The removal of scabrous tissue (debridement) from, for example, a burn is germane to healing. Traditional methods of debridement are mechanical in nature, and involve painful scrubbing, water jets, and rapid movement of liquids. All of these methods have limitations, including patient discomfort, healthy tissue disturbance, and varying degrees of efficacy.

This invention uses the isothermal and isobaric expansion power of a dissolved gas, preferably CO₂, in a liquid to create forces of sufficient magnitude to debride scabrous tissue from a human tissue wound. A liquid containing a dissolved gas is infiltrated into scabrous tissue. A means for activating the system to result in the nucleation and subsequent growth of gas bubbles is provided. Such means can include acoustic (e.g., ultrasonic) energy. Once the system is activated, homogeneous nucleation of gas spontaneously and explosively occurs, resulting in a volumetric expansion that causes scabrous tissue exfoliation. Since the nucleation of the gas is widespread, local stress concentrations are avoided resulting in an essentially pain free event.

The steps for pneumatic debridement are: gas saturation, gas supersaturation, liquid infiltration, activation/nucleation, expansion, and exfoliation. Descriptions for each follow:

Gas saturation—a suitable gas, preferably with high solubility in the infiltrating liquid, is dissolved in the liquid. Carbon dioxide is a desirable gas since it has very high apparent solubility in water. At a pressure (P_{CO2}) of 980 psig, for example, 37 STP volumes of CO₂ will be adsorbed by a water solvent. In beverage industry parlance, 37 volumes of CO₂ are said to be soluble in water under these conditions.

The relationship between P_{CO2} and the amount of CO₂ dissolved in water [CO₂] does not strictly follow Henry's law because CO₂ forms carbonic acid in water, via:

CO₂ (g)+H₂O (l)<=>H₂CO₃

During adsorption at elevated pressure, a quantity of CO₂ is dissolved by the water solvent as a solute. Carbonic acid represents a significant sink for CO₂ as it forms by the reversible reaction previously shown. pH can be used to track CO₂ for control purposes. Both pH and the number of volumes of CO₂ capable of being dissolved are correlated to P_{CO2} in FIG. 1.

Gas Supersaturation—The saturated liquid-gas solution described in the previous section contains 37 STP volumes of CO₂ solute and is represented by Point A in FIG. 1. If the solution is carefully depressurized in a substantially isothermal manner to 1 atmosphere absolute without the nucleation of gas bubbles, it will remain in a supersaturated state.

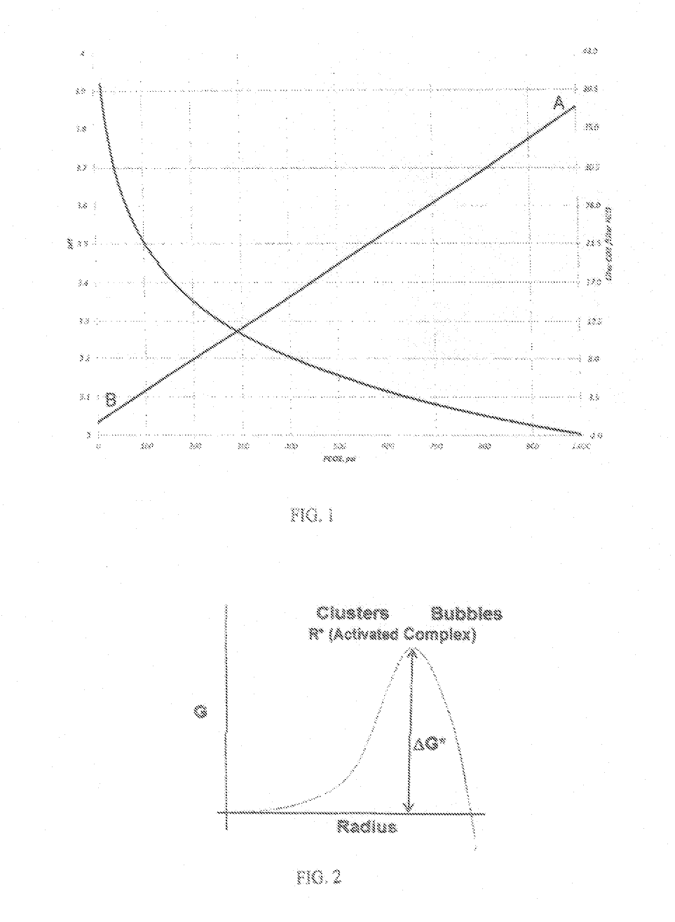

It has been found that if such a liquid is substantially free of suspended particulate and not subjected to mechanical agitation or a rapid temperature and/or pressure change, nucleation of gas bubbles can be substantially avoided and the liquid will become supersaturated at atmospheric pressure and is metastable at this pressure. This supersaturated condition is represented by Point B in FIG. 1. A graphical depiction of a metastable state can be illustrated by a plot of free energy (G) vs. the radii of bubbles to be nucleated as per accompanying FIG. 2.

At bubble radii less than the critical value designated by R*, the solute gas (CO₂) molecules will remain as clusters, be sub-embryonic, metastable, and will not spontaneously nucleate. At R*, however, molecular clusters of CO₂ can reduce their free energy by homogeneous nucleation and subsequent growth. As the bubble radius increases beyond the threshold value of R*, free energy decreases as bubble radius increases and the system is in a stable growth mode. When a subcritical radius exists, supplying energy equivalent to DG* will allow the system to reach the energetic state of an activated complex resulting in the spontaneous homogeneous nucleation of bubbles. Solute gas is subsequently lost to the atmosphere with the release of energy as bubble growth and separation from the liquid solvent phase occurs.

The system will remain in a sub-embryonic state unless activation energy is imparted or particles are present that provoke heterogeneous nucleation. Importantly, metastable gas supersaturation is accomplished by reducing P_{CO2} to an ambient value less than the saturation pressure that was initially used to dissolve the gas without nucleating bubbles. This solution will remain supersaturated provided that pressure reduction from the dissolution pressure to ambient pressure is carefully managed and substantially isothermal.

Liquid Infiltration—Scabrous tissue is topical to nascent tissue as a cut (laceration) wound or burn heals. Such tissue is typically lamellar in its morphology and somewhat porous. A discernible boundary typically exists between the scabrous tissue and underlying nascent tissue. In this development, supersaturated homogeneous solution, as previously described, is infiltrated into the scabrous tissue to be debrided. The well-known mechanism of capillary conduction with hydrophilic surfaces and low viscosity of water facilitates rapid infiltration.

In certain situations where a surface may not be sufficiently hydrophilic, various wetting agents, such as propylene glycol or sodium lauryl sulfate, can be used. Scabrous tissue on human skin should be sufficiently hydrophilic, however, to provide excellent infiltration characteristics.

Supersaturated solution is preferably infiltrated by direct immersion of the site to be debrided. Such immersion can, and preferably should, be full body in nature.

The concentration of dissolved CO₂ may need to be limited in cases involving full body immersion because the solution pH decreases with increasing CO₂ concentration. pH tolerance of an extremity would be expected to be greater.

Full infiltration of supersaturated solution into the scabrous tissue should require minutes. Very mild circulation may reduce infiltration time if performed without turbulence that would provoke gas dissolution. Heating of the solution is also possible if performed at a low rate.

Activation/nucleation—The infiltrated supersaturated CO₂/water solution previously described contains 37 STP volumes of CO₂. If properly activated, the solution will yield gas on a 37:1 gas/liquid volume ratio. Activation supplies the necessary energy (DG*) for the system to reach an activated complex and subsequently nucleate gas. Activation energy will bring the system to an activated complex represented by the peak designated by R*. Once an activated complex is reached, the system will spontaneously liberate stored energy in the dissolved gas phase. This energy liberation is represented as the system passes to the right of R* in accompanying FIG. 2.

Several means of supplying activation energy can be used. The preferred method is to impart mechanical energy in the form of acoustic/ultrasonic energy for inducing local vibrations in the scabrous tissue. This energy can be conveniently supplied by an immersible ultrasonic transducer brought proximate to the wound site. Ensonifying frequencies ranging from, for example, 30 kHz to over 500 kHz can be used for activation. Once the transducer is energized, the resulting vibration will immediately bring the system to an activated complex and gas bubble nucleation will occur.

An alternative to using a separate insertable transducer is to integrate transducers into the sidewall of a container. Other means for activation include a rapid local temperature change induced by a heating probe or rapidly applying a reduced pressure to the same. Heterogeneous nucleation brought about by the introduction of appropriately sized particulate can also be used. In certain situations, it is contemplated that a combination of dispersed particles and ultrasonic energy could be used with the dispersed particles acting as receptors to augment acoustically induced liquid movement. Still further, particulate functioning as receptors for electrical induction energy could provide a local temperature increase of sufficient rate to achieve an activated complex.

Expansion—The nucleation of dissolved solute gas from liquid phase creates a significant volume change. In the case of the CO₂ solution previously cited, a volumetric expansion ratio of 37:1 will occur. If this expansion is substantially isobaric and isothermal, energy availability can be expressed as:

dU=nRT∫_{V1}^{V2}dV/V,

which becomes:

U=nRT ln(V2/V1)

Where:

U=energy

n=number of moles

R=Gas Constant

T=Temperature (abs)

V₁, V₂=initial and endpoint volumes

If, for example, a net 10 cm³ of solution supersaturated to the 37 volume level was infiltrated into scabrous tissue and acoustically activated:

n=37×0.001 Liters×1 mole/22.4 Liters=0.0017 moles

R=8.31 joules/mole-° K

T=310° K

V₁=10 cm³, V₂=370 cm³

U=0.0017 moles×8.31 joules/mole-° K×310° K×ln(37)

U=15.8 joules

If an isothermal expansion occurs over a 10 millisecond time interval, the power released is 15.8 joules/0.01 seconds=1581 watts.

Exfoliation—The energy release calculated in the previous example results in exfoliation of the loose scabrous tissue. This process can be repeated in its entirety to maximize efficacy.

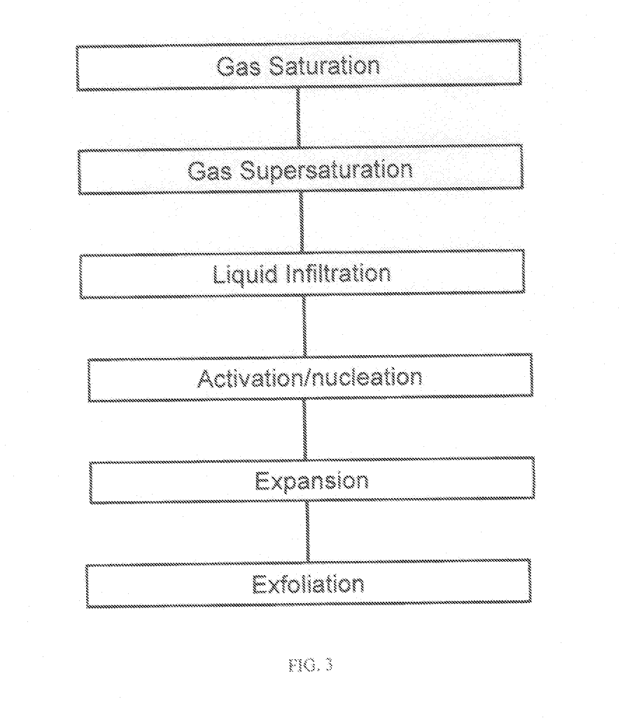

The process steps required for pneumatic debridement are summarized in accompanying FIG. 3. That flowchart depicts the steps necessary to accomplish debridement with carbon dioxide gas according to this invention. Still other gas/liquid combinations can be used, with exfoliation benefit being maximized with such combinations that have the highest solubility of gas in the liquid phase. Oxygen and water is one combination that could also provide other medical benefits as a source of oxygen. Nitrous oxide has been shown to stimulate VEGEF expression that stimulates tissue growth and angiogenesis. Also, nitric oxide has been shown to have certain analgesic effects.

Still other solvent liquids such as ethanol could be used in both medical and non-medical applications provided a specific gas/liquid combination does not create a chemical reaction hazard. Ethanol can provide astringent and disinfectant benefits in medical applications. Additionally, ethanol and other organic solvents can facilitate stripping effects in non-medical applications such as paint removal.

Conjugate treatments involving several gases in sequence or simultaneously are possible. For example, a burn can first be debrided using a CO₂/water combination, followed by an O₂/water combination for oxygen infusion to enhance neoepithelial tissue growth, and concluded with a N₂O/water combination for VEGF expression.

It can be seen from the previous energetic relationship that gases with the greatest number of moles (n) per unit volume are capable of the greatest energy release during expansion. Gases with low density should provide greater energetic benefit on a mass basis than high density gases since a greater number of moles per unit mass will participate in the expansion event.

Since all interphase interfaces have a characteristic surface energy, the creation of interfacial (surface) area is an energetic process. As a gas passes through an orifice, for example, pressure energy is converted to kinetic energy, which consequently satisfies the energetic requirements of the system for the production of surface area. Area and velocity are inversely proportional. Hence, as orifice diameter decreases, the corresponding pressure drop and gas velocity increase, and more surface area are generated. Smaller bubbles result. This process has a limiting condition in that the amount of heat produced (as irreversible work) is inversely proportional to the square of orifice diameter. It therefore becomes impractical and energetically inefficient to operate at exceptionally small orifice diameters. This process also has an absolute limit as a gas velocity of Mach one is approached within the pore of a porous medium used to create bubbles. Because a pore lacks the convergent/divergent geometry required to achieve supersonic flow, increasing pressure beyond the critical pressure will not result in a further reduction of bubble size.

Other methodologies have been used to prepare gaseous solutions based on pressure tanks and adaptations of carbonator devices that dissolve carbon dioxide in water. For a given pressure and temperature, the solubility of carbon dioxide in water exceeds that of oxygen by over an order of magnitude. Carbonators therefore may be acceptable for preparing carbonated water solutions but not oxygenated solutions.

In this invention, conditions most favorable to produce a dispersion of small diameter microbubbles in a suspending solvent liquid with high interfacial area are created either at elevated pressure or with a subsequent increase of pressure. The elevated pressure environment will dissolve the gas in the liquid, since the concentration of a gas in solution and the pressure over the solution is directly related.

High interfacial area enhances the kinetics of the dissolution process, and pressure establishes the maximum concentration of the gas held in solution. For wellness applications, oxygen may be intentionally nucleated from an aqueous oxygen solution to form a dispersion of microbubbles held within the solution and capable of providing the benefits of pure oxygen. Living tissue, and in particular, human skin exposed to such an environment will have the opportunity for oxygen uptake both from the suspension of pure oxygen microbubbles and oxygen adsorbed from the liquid solution at near atmospheric pressure.

Having described the presently preferred embodiments, it is to be understood that the invention may be otherwise embodied within the scope of the appended claims.

What is claimed is:

1. A method for debriding human tissue, said method comprising:
(a) dissolving a gas in a liquid to form a solution under elevated pressure for saturating said solution with the dissolved gas;
(b) depressurizing said solution at or towards ambient pressure for supersaturating the dissolved gas therein;
(c) providing means for allowing said supersaturated solution to infiltrate said human tissue to be debrided;
(d) activating said dissolved gas in said supersaturated solution;
(e) allowing said activated gas to expand for releasing energy into the solution at or near said human tissue to be debrided; and
(f) exfoliating said human tissue to be debrided with said released energy.

2. The method of claim 1 wherein said human tissue to be debrided consists of one or more lacerations.

3. The method of claim 1 wherein said human tissue to be debrided consists of one or more burns.

4. The method of claim 1 wherein said human tissue to be debrided consists of one or more scabrous regions.

5. The method of claim 1 wherein said human tissue to be debrided consists of a skin disease.

6. The method of claim 1 wherein the gas of step (a) consists essentially of carbon dioxide.

7. The method of claim 1 wherein the solution of step (b) is substantially free of suspended particulates.

8. The method of claim 1 wherein depressurizing step (b) occurs without mechanical agitation.

9. The method of claim 1 wherein depressurizing step (b) occurs without rapid temperature or pressure changes.

10. The method of claim 1 wherein contacting step (c) includes fully immersing said human tissue in said supersaturated solution.

11. The method of claim 1 wherein contacting step (c) includes adding a wetting agent.

12. The method of claim 1 wherein activating step (d) includes introducing acoustic energy into said solution.

13. The method of claim 12 wherein activating step (d) includes integrating one or more acoustic transducers into a container for said solution.

14. The method of claim 1 wherein debriding is accompanied by exposing the human tissue to a solution containing at least one dissolved gas selected from the group consisting of:
oxygen, nitrous oxide, nitric oxide, carbon monoxide and hydrogen sulfide.

15. A method for pneumatically debriding a burn wound to human tissue, said method comprising:
(a) dissolving carbon dioxide gas in a liquid to form a solution under elevated pressure for saturating said solution with the dissolved gas;
(b) depressurizing said solution at or towards atmospheric pressure for supersaturating the dissolved gas therein;
(c) immersing said burn wound in said supersaturated solution;
(d) activating said dissolved gas in said solution;
(e) allowing said activated gas to expand for releasing energy into the solution at or near said burn wound; and
(f) exfoliating said burn wound with said released energy.

16. The method of claim 15 wherein the solution of step (b) is substantially free of suspended particulates.

17. The method of claim 15 wherein depressurizing step (b) occurs without mechanical agitation, rapid temperature or pressure changes.

18. The method of claim 15 wherein activating step (d) includes introducing acoustic energy into said solution.

19. The method of claim 18 wherein activating step (d) includes integrating at least one acoustic transducer into a container for said solution.

20. The method of claim 15 wherein debriding is accompanied by exposing the burn wound to a solution containing at least one dissolved gas selected from the group consisting of: oxygen, nitrous oxide, nitric oxide, carbon monoxide and hydrogen sulfide.

21. A method for pneumatically debriding a laceration to human tissue, said method comprising:
(a) dissolving carbon dioxide gas in a liquid to form a solution under elevated pressure for saturating said solution with the dissolved gas;
(b) depressurizing said solution at or towards atmospheric pressure for supersaturating the dissolved gas therein;
(c) immersing said laceration in said supersaturated solution;
(d) activating said dissolved gas in said solution;
(e) allowing said activated gas to expand for releasing energy into the solution at or near said laceration; and
(f) exfoliating said laceration with said released energy.

22. The method of claim 21 wherein the solution of step (b) is substantially free of suspended particulates.

23. The method of claim 21 wherein depressurizing step (b) occurs without mechanical agitation, rapid temperature or pressure changes.

24. The method of claim 21 wherein activating step (d) includes introducing acoustic energy into said solution.

25. The method of claim 24 wherein activating step (d) includes integrating at least one acoustic transducer into a container for said solution.

26. The method of claim 21 wherein debriding is accompanied by exposing the laceration to a solution containing at least one dissolved gas selected from the group consisting of: oxygen, nitrous oxide, nitric oxide, carbon monoxide and hydrogen sulfide.
